(19) United States
(12) Patent Application Publication
(10) Pub. No.: US 2008/0118990 A1
(43) Pub. Date: May 22, 2008

(54) Assay For Lipoproteins Using Lumiphore K-37

(75) Inventors: Gareth Royston Jones, Altrincham (GB); David Thomas Clarke, Warrington (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Science and Technology Facilities Council, Warrington (GB)

(21) Appl. No.: 11/792,874

(22) PCT Filed: Dec. 12, 2005
(86) PCT No.: PCT/GB05/04794

§ 371 Date: Jun. 7, 2007

(30) Foreign Application Priority Data
Dec. 11, 2004 (GB) 0427191.2

(51) Int. Cl. G01N 33/566; G01N 21/76

(52) U.S. Cl. 436/501; 422/52

(57) ABSTRACT

The present invention relates to a method of determining the concentration of total lipoprotein in sample solution. The method comprising the steps of adding to an aliquot of the sample a probe substance, K-37 which binds to lipoproteins in the sample, and which when so bound fluoresces under appropriate excitation. The total lipoprotein concentration in the sample is then determined using fluorescence analysis. The method may employ a second probe substance that is specific to a class or sub-class of lipoproteins in order that the user may distinguish between lipoproteins. The invention further relates to apparatus which may be used to perform the methods of the invention.

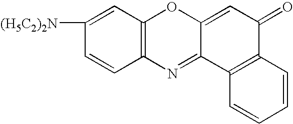

The present invention relates to an assay system for discriminating between different classes of lipid molecules in a sample mixture. In particular, the invention relates to a method of determining the concentration of particular lipoproteins in blood plasma or serum. The invention further relates to an apparatus for carrying out the method.

Lipids are a diverse group of organic compounds occurring in living organisms. They are insoluble in water, but soluble in organic solvents. Lipids are broadly classified in to two categories: (i) complex lipids; and (ii) simple lipids. Complex lipids are esters of long-chain fatty acids and include glycerides, glycolipids, phospholipids, cholesterol esters and waxes. Simple lipids, which do not contain fatty acids, include steroids (for example, cholesterol) and terpenes.

Lipids can combine with proteins to form lipoproteins, which is the form in which lipids, such as cholesterol and triglycerides, are transported in blood and lymph. The lipoproteins found in blood plasma fall into three main classifications: (i) high density lipoproteins (HDL), (ii) low density lipoproteins (LDL), and (iii) very low density lipoproteins (VLDL), together with intermediate density lipoproteins (IDL). For brevity, the term “serum” is used herein, but references to “serum” should be interpreted as references to serum or plasma.

It is well documented that there is a strong relationship between the concentration of the various lipoproteins in blood plasma and the risk of atherosclerosis, i.e. the development of harmful plaques on blood vessel walls, which can lead to a heart attack. It is also known that the different classes of lipoproteins (HDL, LDL and VLDL) each play a different role in atherosclerosis. For instance, HDL is regarded as being anti-atherogenic whereas LDL is known to be highly atherogenic (the cholesterol it carries correlating closely with atheroscleroses development).

Therefore, knowledge of the relative concentrations of each of the various lipid components in the blood (i.e. the lipoproteins) would be advantageous, as this would assist a clinician in treating patients having blood concentrations of these lipids, which are inappropriate. It will be appreciated that having a knowledge of the patient's lipoprotein profile would be most advantageous to the clinician.

Assays have been developed for determining the concentrations of some of the lipid components in blood. Such assays normally involve initially taking a blood sample from a patient, which is then sent to a clinical laboratory for analysis. Such assays are carried out using expensive equipment and for logistical reasons a considerable length of time is taken to generate results. This delays treatment. Furthermore, the tests are involved and are therefore expensive. In addition, the equipment used in the lab is not readily portable and so cannot be used by GPs, or nurses, carrying out house calls, or even as test kits for home use. Devices have recently been developed that attempt to reproduce lab assays at “point of care” but these have proved to be expensive and require an expert user to operate. Accordingly, there is a requirement to provide improved methods for analysing the lipoprotein profile in blood serum.

Blood serum is a complex mixture of a variety of proteins, and although methods for separating and directly measuring the concentration of the different classes of lipoproteins are known, such methods are complex and expensive. An example of an assay for determining the lipoprotein concentration of blood serum is disclosed in WO 01/53829A1. This document relates to the use of a particular organic luminophore, 4-dimethylamino-4′-difluoromethyl-sulphonyl-benzylidene-acetophenone (DMSBA), as a fluorescent probe. The formula of the probe, identified as K-37, is given below:

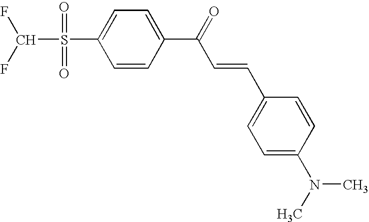

The probe K-37 is not luminous in water, but is highly luminous in aqueous lipoprotein solutions, such as blood serum. In particular, the intensity of the fluorescence is highly dependent upon the lipoprotein content of the blood serum and thus K-37 can be used as a fluorescent probe to measure the concentration of lipoproteins that may be present, i.e. K-37 fluoresces when bound to the lipids of lipoproteins and is excited at appropriate radiation wavelengths. Accordingly, measurement of the time-resolved fluorescence decay of a lipoprotein mixture can be used to give direct information as to the relative concentrations of the different lipoproteins (LDL, and VLDL) present in that mixture.

However, problems with using K-37 time-resolved fluorescence decay is that its measurement is complex and requires expensive equipment. Furthermore, it involves highly technical computer analysis of the data produced, which can be time-consuming to interpret correctly. Accordingly, use of K-37 time-resolved fluorescence decay to determine the concentrations of lipid components in blood has serious limitations for a clinician when wishing to quickly decide a course of treatment rather than taking the time to use time-resolved fluorescence decay to provide a lipoprotein analysis.

Therefore, even though there are methods available for determining the concentration of specific lipoproteins in a sample, by using time-resolved fluorescence analysis with the probe K-37, it will be appreciated that this method has a number of limitations.

It is therefore an aim of embodiments of the present invention to obviate or mitigate the problems with the prior art, and to provide an improved method for determining the concentration of lipoproteins in a sample. It is a further aim to provide an apparatus for carrying out said method.

The inventors have developed a simplified assay based on the use of K-37 for measuring lipoproteins in a biological molecule. For determining the concentration of total lipoprotein (i.e. HDL, LDL, and VLDL) in a blood sample using K-37 fluorescence measurements, the inventors realised that it would be preferred that the fluorescence response from the probe substance bound to the various lipoprotein classes must be substantially the same for a given total lipoprotein concentration, i.e. total lipoprotein concentration, irrespective of its composition (i.e. the ratio of HDL:LDL:IDL:VLDL in the sample). Accordingly, the inventors believed that it would be preferred that the response of fluorescence intensity from the probe substance should also be substantially linear across the range of concentrations of lipoprotein molecules that would be expected from samples that would be encountered in clinical tests.

While the inventors do not wish to be bound by any hypothesis, they believe that the intensity of fluorescence from the probe substance will depend on its affinity for a particular lipoprotein molecule (HDL, LDL, IDL or VLDL) in the sample, the quantum yield of fluorescence depending on the environment within that lipoprotein molecular complex, and also the degree of fluorescence quenching caused by energy transfer between probe molecules packed closely together. Hence, the inventors reasoned that it may be possible to select a suitable concentration of the probe substance that may be used to make an accurate measurement of total lipoprotein by simple fluorescent measurement. The inventors further realised that such a concentration of probe would need to balance K-37 's higher quantum yield in HDL compared to VLDL and LDL with its higher affinity for HDL, and therefore a higher degree of quenching within HDL to produce a constant fluorescence signal response over all lipoprotein particles.

The inventors therefore conducted a series of experiments (discussed in Example 1) to investigate whether it was possible to obtain a linear and equal relationship between the fluorescence of the probe substance, K-37, and the lipoprotein concentration for each lipoprotein particle type (HDL, LDL, and VLDL), across the range of lipoprotein concentrations that would be encountered in real serum samples. To their surprise, they found that there was a defined concentration of K-37 at which there was a linear relationship between the fluorescence of K-37 and lipoprotein concentration.

Hence, according to a first aspect of the present invention, there is provided a method of determining the concentration of total lipoprotein in sample solution, the method comprising the steps of:
(i) adding to an aliquot of the sample between 0.1 mM-1.0 mM of a probe substance, K-37 (as defined herein), which probe substance binds to lipoproteins in the sample, and which when so bound fluoresces under appropriate excitation; and
(ii) determining the total lipoprotein concentration in the sample using fluorescence analysis.

By the term “total lipoprotein”, we mean the collective concentration of VLDL, HDL, LDL, IDL and chylomicrons in the sample.

Advantageously, at the concentration of 0.1-1.0 mM K-37, a more accurate determination of the concentration of the total lipoprotein is possible, as there is surprisingly considerably less signal distortion obtained from analysis of the fluorescence measurement of step (ii) of the method.

Suitably, the concentration of K-37 added to the sample may be between approximately 0.2-1.0 mM, more suitably, between approximately 0.3-0.9 mM, and even more suitably, between approximately 0.5-0.8 mM. Preferably, the concentration of K-37 added to the sample is between approximately 0.65-0.75 mM. 0.65 mM K-37 is a preferred concentration and an especially preferred concentration is about 0.7 mM K-37.

Hence, in a preferred embodiment, approximately 0.7 mM of the probe substance, K-37, is added to the sample in step (i) of the method in order to carry out step (ii) of the method according to the invention.

The inventors have found that 0.65 mM K-37 is useful in a number of experimental conditions although 0.7 mM represents the preferred concentration of this probe when biological samples, which contain proteins, are assayed.

Preferably, the method according to the first aspect comprises exciting the sample at an excitation wavelength of between about 400 nm-500nm, and more preferably, between about 420 nm-480 nm, and even more preferably, between about 440 nm-470 nm. An especially preferred excitation wavelength of about 450 nm may be used although excitation at any wavelength between about 450-470 nM is also particularly preferred.

Preferably, the method comprises observing the fluorescence at an emission wavelength of between about 500-650 nm, and more preferably, between about 520 nm-600 nm. An especially preferred emission wavelength of about 540 nm (or higher) may be used, at which the most accurate readings for determining the total lipoprotein concentration (i.e. the concentration of HDL, IDL, LDL and VLDL, but also chylomicrons if present) may be observed.

By the term “fluorescence analysis”, we mean the measurement of fluorescence of the products of the lipoprotein assay, by first exciting the sample so that it fluoresces, and then observing the fluorescence.

The sample may be a foodstuff, for which knowledge of the total lipoprotein concentration therein is required. Preferably, the sample is a biological sample, which may be obtained from a subject to be tested. The sample may comprise any biological fluid, for example, blood plasma or serum, or lymph. It is especially preferred that the sample comprises blood serum or plasma.

The inventors realised that the lipoprotein profile that may be generated using the method according to the invention may be further improved and more detailed, if they could distinguish between the various lipoproteins in the sample being tested. Therefore, the inventors investigated the use of probe substances other than K-37 to see if it was possible to distinguish between the various lipoprotein molecules. They were surprised to find that a number of dyes are available that will bind to lipoproteins and will exhibit different fluorescent responses that are dependant on the particular lipoprotein bound. Fluorescent measurements with these dyes makes it possible to distinguish between the types of lipoprotein present in a sample. This is done by comparing the enhanced or reduced fluorescence caused by one type of lipoprotein in a lipoprotein mixture with the fluorescence expected from the other lipoproteins (in the absence of the specific propertied lipoprotein) as determined from a calibration curve and a known value of the total lipoprotein content given by the K-37 assay. For example the fluorescent dye, Nile Red, exhibited a significantly higher fluorescence in HDL than in the other lipoproteins, such as LDL and VLDL. Therefore, the inventors realised that a second probe substance (e.g. Nile Red, or any other lipophilic probe that shows specificity, or fluorescence enhancement or reduction towards a particular lipoprotein), may be used to discriminate between classes or subclasses of lipoproteins in the sample. This is possible after the total lipid concentration has been determined.

Accordingly the method of the invention may further comprise determining the concentration of a particular class, or sub-class of lipoprotein by the shift in fluorescence response of the dye specific to that lipoprotein using a second probe. The second probe substance may be added to a second aliquot of the sample, which probe binds to a specific class or subclass of lipoproteins and which when bound thereto, modifies the fluorescence yield under appropriate excitation, which is indicative of the concentration of the specific class or sub-class of lipoproteins.

It is preferred that this further steps comprises adding the probe Nile Red to a separate aliquot of the sample after step (i) of the method, in order to assay for HDL in the sample. This is described in detail in Example 3.

Preferably, in order to determine the HDL concentration in the sample using Nile Red, a calculation must be made of the excess fluorescence from Nile Red due to the presence of HDL. Firstly, the total lipoprotein concentration (measurement “A”) is measured by the linear correlation of K-37 fluorescence with lipoprotein concentration (as determined by step (ii)).

Secondly, Nile Red fluorescence is then calibrated with LDL (and/or VLDL as the fluorescence to concentration response must be essentially the same) at various concentrations to obtain a calibration curve with slope “X” and intercept “Y”. A skilled technician would know how to prepare a range of concentrations of LDL (and/or VLDL), and determine the respective fluorescence for each concentration.

Thirdly, an additional calibration curve is then constructed for a series of concentrations of HDL and a constant concentration of LDL to give slope “Z”. Fourthly, knowing the total lipoprotein concentration from the K-37 measurement “A” and the excess Nile Red fluorescence of the unknown sample “B”, the concentration of HDL “C” in the unknown sample can be determined by the following equation

C=(B−(AX−Y))/Z

It will be appreciated that in the practice of the invention that pre-prepared or standard calibration curves may be used. Furthermore devices developed to generate lipid profiles (see below) may have internal standards and/or have processing means that will allow for automatic calculation of HDL levels without user intervention.

Therefore, the method according to the invention may further comprise determining the concentration of HDL in the sample using fluorescence analysis. The method comprises a further step in which the probe substance Nile Red is added to a second aliquot of the sample, which probe binds to HDL and other lipoproteins. Under appropriate excitation Nile Red fluoresces more and more strongly in proportion to the concentration of HDL in the sample. When this additional step is carried out in the method of the invention, an even more detailed lipoprotein profile of the sample may be generated consisting of total lipoprotein concentration, and HDL concentration, which would be very useful to the clinician.

The inventors conducted a series of experiments to determine the optimum concentration of Nile Red, which should be added to the sample, to improve the accuracy of the determination of HDL in the sample, and this required considerable inventive endeavour. Accordingly, the concentration of the probe substance Nile Red added to the sample may be between approximately 0.1-1 mM. Advantageously, at this concentration of Nile Red, a more accurate determination of the concentration of the HDL concentration is possible.

Suitably, the concentration of Nile Red added to the sample may be between approximately 0.1-0.9 mM, more suitably, between approximately 0.2-0.7 mM, and even more suitably, between approximately 0.3-0.6 mM. It is especially preferred to add Nile Red to the sample to a final concentration of about 0.4 mM.

The fluorescence of Nile Red is preferably induced by exciting the sample at an excitation wavelength of between about 400 nm-650 nm.

It is preferred that the excitation wavelength is 400 nm-650 nm; preferably, between about 420 nm-620 nm, more preferably, between about 500 nm-610 nm and even more preferably, between about 590 nm-610 nm. An excitation wavelength of about 600 nm may be used in connection with Nile Red which gives the largest discrimination (5×) between the fluorescence response from Nile Red in HDL when compared with the other lipoproteins. When these excitation wavelengths are employed it is preferred that an agent is used that blocks the “fatty acid and drug binding domain” on Human Serum Albumin (HSA) as discussed in more detail below.

The resultant fluorescence from Nile Red may then be observed and measured at an emission wavelength of between about 540-700 nm, and more preferably, between about 570-650 nm. A preferred emission wavelength of about 620 nm may be used, at which the most accurate readings for determining the concentration of HDL may be observed.

The inventors investigated whether it was possible to further improve the accuracy of the individual assays used in the method according to the invention, and so turned their attention to Human Serum Albumin (HSA), which is a major component of blood serum, having a concentration of approximately 30-50 mg/ml.

HSA is known to have at least two types of binding site that are capable of binding various ligands. A first type is referred to herein as “a hydrophobic domain” whereas a second type of domain is referred to herein as a “drug binding domains”. These domains are known to one skilled in the art and are distinguished from each other in a paper in Nature Structural Biology (V5 p827 (1998)). This paper identifies the hydrophobic domain as one to which fatty acids may bind whereas the drug binding domain is capable of binding a number of drugs that may be associated with HSA.

From their experiments, the inventors have surprisingly established that the probe substances K-37 and Nile Red may both individually bind to hydrophobic binding sites/domains of HSA. Hence, K-37 and Nile Red are both ligands for HSA. In addition, surprisingly, the inventors found that K-37 and Nile Red fluoresce when bound to HSA. Therefore, while the inventors do not wish to be bound by any hypothesis, the inventors believe that this additional fluorescence of K-37 when bound to HSA may cause a substantial background signal, which may distort and lead to significant errors in the determination of concentration of total lipoprotein in step (ii) of the method according to the invention. Similarly, the inventors believe that this additional fluorescence of Nile Red when bound to HSA may cause a substantial background signal, which may distort and lead to significant errors in the determination of concentration of HDL when this additional step is used in the method according to the invention.

As a result, the inventors investigated the effects of inhibiting the binding of the ligand K-37, and the ligand Nile Red, with HSA. In particular, they attempted to block the hydrophobic binding sites of HSA at which the probes K-37 and Nile Red bind and fluoresce. This work is described in Examples 3 and 4. While the inventors do not wish to be bound by any hypothesis, to their surprise, they found that inhibiting the binding of the ligand K-37 with the hydrophobic binding sites resulted in the fluorescence of the probe substance when bound to the lipoprotein molecules (HDL, LDL, VLDL) being a more accurate measure of the concentration of total lipoprotein in the sample than if no ligand binding inhibitor was added. The inventors also found that inhibiting binding of the ligand Nile Red to HSA improved the accuracy of the HDL determination.

Accordingly, it is preferred that the method according to the invention comprises adding to the sample a ligand binding inhibitor that is adapted to substantially inhibit the binding of the probe substance (K-37 and/or Nile Red) to HSA, preferably, the hydrophobic binding sites thereof. It is especially preferred that the ligand binding inhibitor is also added to the sample prior to or at the same time as step (i). It is also preferred that the inhibitor is added to the separate aliquot to which Nile Red is added, for carrying out the HDL assay.

The ligand binding inhibitor may be hydrophobic. The inhibitor may be amphipathic. The ligand binding inhibitor may comprise a fatty acid or a functional derivative thereof, as well as other hydrophobic molecules. Examples of suitable derivatives of fatty acid, which may block the hydrophobic binding sites of HSA may comprise a fatty acid, its esters, acyl halide, carboxylic anhydride, or amide etc. A preferred fatty acid derivative is a fatty acid ester.

The fatty acid or derivative thereof may comprise a C₁-C₂₀ fatty acid or derivative thereof. It is preferred that the fatty acid or derivative thereof may comprise a C₃-C₁₈ fatty acid or derivative thereof, more preferably, a C₅-C₁₄ fatty acid or derivative thereof, and even more preferably, a C₇-C₉ fatty acid or derivative thereof.

It is especially preferred that the ligand binding inhibitor comprises octanoic acid (C₈) or a derivative thereof, for example, octanoate. Preferably, the ligand binding inhibitor is added as an alkali metal octanoate, preferably a Group I alkali metal octanoate, for example, sodium or potassium octanoate.

Preferably, between about 10-400 mM of the ligand binding inhibitor is added to the sample prior to carrying out step (i) of the method, more preferably, between about 20-200 mM, and even more preferably, between about 50-150 mM is added. It is especially preferred that about 100 mM of the inhibitor is added. Hence, in a preferred embodiment of the method, about 100 mM of sodium octanoate may be added to the sample before or at the same time as carrying out step (i).

When the method of the invention also extends to the use of Nile Red, it is preferred that between about 10-400 mM of the ligand binding inhibitor is added to the sample, more preferably, between about 20-200 mM, and even more preferably, between about 50-150 mM is added. It is most preferred that about 100 nM of the inhibitor is used. Hence, in a preferred embodiment of the method, it is preferred that about 100 mM of sodium octanoate is added to the sample before or at the same time as adding Nile Red and carrying out the HDL assay according to the method.

In a preferred embodiment of the invention, a ligand binding inhibitor, for example, about 100 mM sodium octanoate, is first added to an aliquot taken from the sample, with approximately 0.7 mM of the K-37 probe, prior to carrying out the fluorescence measurement of the total lipoprotein concentration in step (i) of the method. In another embodiment, a ligand binding inhibitor, for example, about 100 mM sodium octanoate, is first added to a further aliquot, with approximately 0.4 mM of the Nile Red probe, prior to carrying out the fluorescence measurement of the HDL concentration in the method.

Advantageously, the ligand binding inhibitor combined with the defined concentration of the K-37 probe result in highly accurate measurements of total lipoprotein being obtained (and HDL concentration when Nile Red probe is added).

Examples 1 and 3 illustrate how fluorescence measurements of the dye K37 may be used to determine the concentration of total lipoproteins in the sample, and how the fluorescence measurements of Nile Red may be used to determine the concentration of HDL in the sample. The examples also describe blocking HSA with a ligand binding inhibitor in order to optimise the accuracy of the results produced by K-37 or Nile Red fluorescence. Therefore, the inventors realised that it is possible to create a single parallel method for analysing the lipoprotein composition.

Hence, a preferred method consists of two assays (total lipoprotein and HDL), which produce results almost instantaneously. The clinician may use this information to decide upon a certain course of treatment.

The inventors have additionally found that Nile Red also interacts with the drug binding domain on HSA that is referred to above. Ligands for this drug binding domain include drug molecules such as: thyroxine, ibuprofen, diazepam, steroid hormones and their derivatives (drugs), haem, bilirubin, lipophilic prodrugs, warfarin, coumarin based drugs, anaesthetics, diazepam, ibuprofen and antidepressants (e.g. thioxanthine).

The inventors have found that agents may be used to block this drug binding domain and that this results in further improvement of assay results with Nile Red. The abovementioned drugs, or any other molecule with affinity to this domain, may be used as agents for blocking the drug binding domain of HSA. However it is most preferred that benzoic acid or a derivative thereof (e.g. trichlorobenzoic acid or triiodobenzoic acid) is used to block the drug binding domain.

Therefore, a most preferred embodiment of the invention may comprise taking a blood sample from a patient, and then separating the blood serum from the blood cells. This may be achieved by known techniques, such as filtration or centrifugation. The plasma may then be separated in to two aliquots, each of which is subjected to biochemical analysis to determine the concentration of a lipid component. A first aliquot may be used to determine the concentration of total lipoprotein in the sample, and a second aliquot may be used to determine the concentration of HDL in the sample knowing the total lipoprotein in the sample.

To the first aliquot, an HSA ligand binding inhibitor, for example, sodium octanoate, may be added. It is preferred that the probe K37 is also added to the first aliquot in step (i), preferably to a final concentration of about 0.7 mM. The first aliquot may then be excited at approximately 450 nm in order to cause the probe to fluoresce. The fluorescence may then be measured at an emission wavelength of 540 nm or above. From this value, it is then possible to determine the concentration of total lipoprotein (HDL, LDL, IDL and VLDL and chylomicrons if present) in the sample.

To the second aliquot, an HSA ligand binding inhibitor for example, sodium octanoate, may be added to a final concentration of about 100 mM. An agent that will bind to the drug binding domain of HSA such as benzoic acid may also be added, to a concentration between 1-10 mM or more specifically approximately 5 mM. The probe Nile Red may then be added to a final concentration of about 0.4 mM. This sample may then be excited at approximately 600 nm in order to cause the probe to fluoresce. The fluorescence may be measured at an emission wavelength of approximately 620 nm, and from this value it is then possible to determine the concentration of HDL in the sample as described in Example 3.

In a most preferred embodiment of the invention the method may comprise:
(A) adding 100 mM sodium octanoate (a ligand binding inhibitor) to a first aliquot taken from a biological sample, with approximately 0.7 mM of the K-37 probe; exciting at about 450 nm; and carrying out a fluorescence measurement, at an emission wavelength of approximately 540 nm, of the total lipoprotein concentration in step (i) of the method; and
(B) adding a 100 mM sodium octanoate (a ligand binding inhibitor)) and 5 mM sodium benzoate (an agent that blocks the drug binding domain of HSA) to a second aliquot taken from the sample, with approximately 0.4 mM of the Nile Red probe; exciting at about 600 nm; and carrying out a fluorescence measurement at an emission wavelength of approximately 620 nm to determine HDL concentration; and
(C) calculating total lipoprotein concentration according to step (ii) of the method of the invention and also HDL concentration.

In addition, to developing the method according to the first aspect, the inventors also developed an apparatus for carrying out the method.

Hence, according to a second aspect of the present invention, there is provided apparatus for determining the concentration of total lipoprotein in a sample, the apparatus comprising a reaction reservoir for conducting a lipoprotein assay; containment means adapted to contain reagents required for the method according to the first aspect; excitation means operable to excite the sample so that it fluoresces, and detection means operable to detect the fluorescence emitted by the sample.

Preferably the apparatus comprises means for mixing the sample and reagents in the reaction reservoir.

Preferably, the apparatus comprises a number of types of reservoir.

A first type of reservoir may be for containing the sample and is referred to herein as a sample reservoir.

The reaction reservoir may be a second type of reservoir in which the assay to determine the concentration of lipoprotein in the sample may be conducted (following introduction of the sample and reagents). The apparatus may comprise a single reaction reservoir and may be washed out between reactions on different sample aliquots. Alternatively multiple (e.g. single use) reaction reservoirs may be brought into contact with the excitation means.

The containment means may comprise a third reservoir (a first reagent reservoir) containing the K-37 dye and other reagents for the total lipoprotein assay (e.g. a ligand binding inhibitor). When HDL is to be determined (e.g. using Nile Red) the containment means may comprise a fourth reservoir (a second reagent reservoir), containing Nile Red dye and other reagents for determining HDL (e.g. a ligand binding inhibitor and an agent for blocking the drug binding domain of HSA). Alternatively the K-37 reagents and Nile Red reagents may be included in separate containment means. Accordingly reagents for the K-37 assay may be within a first reagent reservoir in a first containment means and the reagents for the Nile Red assay may be within second reagent reservoir in a second containment means.

It is preferred that the reaction reservoir is arranged so that it may be brought into optical contact with the excitation means. It is preferred that the reaction reservoir is arranged so that fluorescence produced from the assay may be detected by the detection means.

It will be appreciated that, in some embodiments, the device may be designed such that the sample may be directly introduced into the reaction reservoir. This would obviate the need for a first or sample reservoir.

In a preferred embodiment the first reagent reservoir contains the probe substance, K-37, in a suitable diluent and may further contain an HSA ligand binding inhibitor, for example, sodium octanoate.

The second reagent reservoir may comprise the probe substance Nile Red and optionally an HSA ligand binding inhibitor, for example, sodium octanoate and, also optionally, an agent for blocking a drug binding domain on HSA such as benzoic acid.

The apparatus may comprise a reader and preferably, a cartridge adapted to be placed in functional communication therewith. Preferably, the cartridge may be inserted into, or attached to, the reader. The reader may comprise docking means in which the cartridge is inserted. The docking means may be a slot. Hence, preferably, the cartridge is removable from the reader.

The cartridge may comprise the, or each, containment means and the reaction and sample reservoir (if present). Hence, the cartridge carrying the assay reagents may be removed from the reader once the reagents have been exhausted, and replaced with a new cartridge containing new assay reagents. It will be appreciated that a self-contained cartridge (comprising all reservoirs) may be readily used as a single-use reaction cartridge. A cartridge may be simply removed from the reader and replaced with a new cartridge comprising reagent and sample that may be deployed into a reaction reservoir within the cartridge.

The reader may comprise the excitation means and preferably, the detection means.

Preferably, the apparatus comprises processing means adapted to determine the concentration of total lipoprotein in the sample based on the fluorescence detected. In a preferred embodiment, the processing means is also adapted to determine the concentration of HDL in the sample based on fluorescence analysis. The processing means may be adapted to calculate the concentration of LDL, VLDL and IDL, in the sample based on the concentrations of total lipoprotein, and HDL.

The apparatus may comprise display means for displaying the concentration of total lipoprotein and preferably, the concentration of HDL in the sample, preferably as a read-out. For example, the display means may comprise an LCD screen, or may rely on a computer for powering and/or computing and/or display.

Preferably, the apparatus is portable, and may be used to generate a patient's lipoprotein profile by a taking a sample therefrom. The sample may be any biological fluid, for example, blood, serum, lymph etc.

Preferably, the excitation means comprises an illumination source operable to illuminate the sample at about 400 nm-500 nm (for the K-37 assay) and for preferred embodiments of the Nile Red assay, at about 600 nm. Accordingly the light source is preferably capable of illuminating the sample at between about 400 nm-600 nm. The illumination source may comprise a bulb or one or more LEDs and the excitation wavelengths may be varied utilising a 450 nm interference filter and an interference filter at 600 nm. The excitation means may comprise polarising means operable to polarise light produced by the illumination source. The excitation means may comprise focussing means adapted to focus the light on to the sample. The focussing means may comprise a lens.

Preferably, the detection means comprises a photodiode or photomultiplier, which is preferably yellow-red sensitive. Fluorescence emitted by the sample is preferably detected at about 500 nm-650 nm, and more preferably, 540 nm for K-37 and longer wavelengths. The detection means should be able to detect fluorescence emitted at 620 nm for assays involving Nile Red. The fluorescence may be collected by a second lens, and may pass through a polariser. Scattered excitation light may be removed by a cut-off filter. For measurement of the fluorescence intensity, the current from the photodiode or the count rate from the photomultiplier may be read from an ammeter, voltmeter, or rate-meter module.

In one embodiment, the apparatus may comprise a reader that is adapted to receive two (or more) cartridges. Such a reader may comprise two (or more) excitation means that can be aligned with each reaction reservoir. In addition, the apparatus may comprise a detection means for each reaction reservoir.

The apparatus may also comprise an excitation correction system so that fluctuations of the light source may be accounted for. The apparatus may comprise at least one fluorescence standard for use in calibrating prior to determine the concentration of lipoprotein. The standard may be an internal standard.

Accordingly, the apparatus is configured to detect and measure the fluorescence intensities of each assay simultaneously or in turn as the cartridge enters the reader or at some time thereafter, to thereby generate the lipoprotein profile consisting of total lipoprotein concentration, and HDL concentration.

Advantageously, the apparatus according to the second aspect may be used to carry out quick and easy assays, which can be conducted simultaneously to generate the lipoprotein profile from the biological fluid. A clinician with knowledge of lipoprotein and HDL concentrations can then decide on an effective course of treatment. In addition, the apparatus is portable and may be used by GPs, or nurses who carry out home visits, or even as test kits for home use.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:

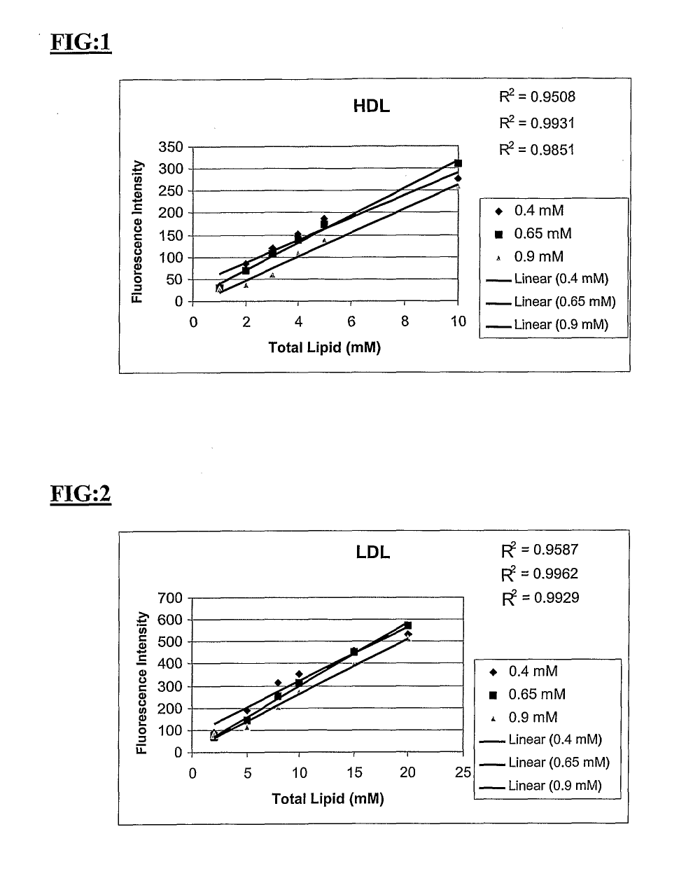

FIG. 1 is a graph showing fluorescence intensity against total lipid concentration for K-37 at three concentrations (0.4 mM, 0.65 mM and 0.9 mM) in HDL as referred to in Example 1;

FIG. 2 is a graph showing fluorescence intensity against total lipid concentration for K-37 at three concentrations (0.4 mM, 0.65 mM and 0.9 mM) in LDL as referred to in Example 1;

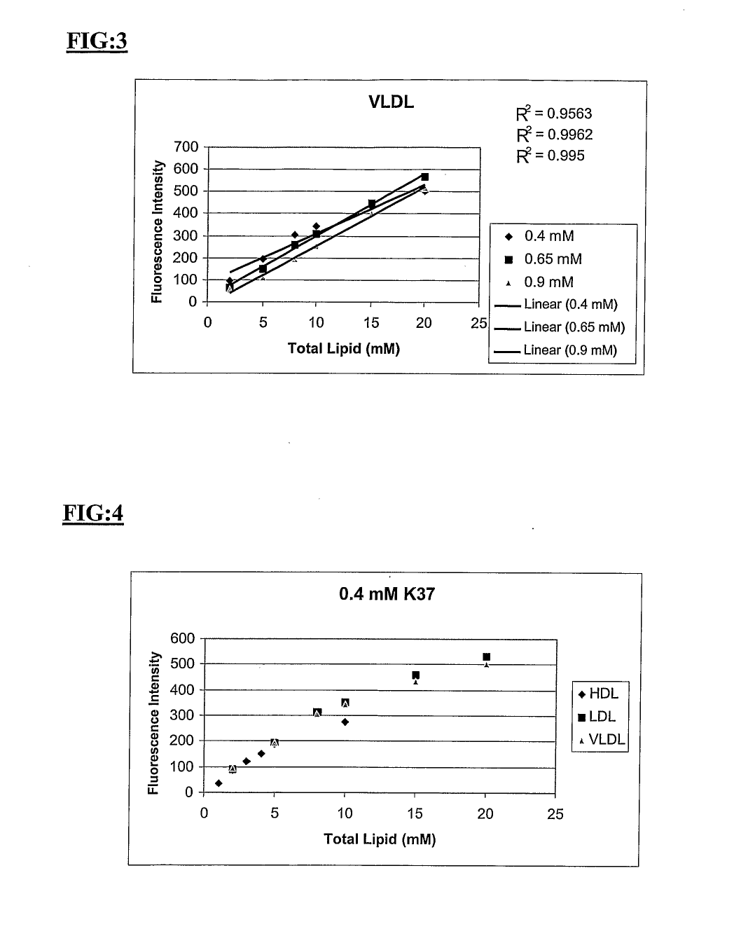

FIG. 3 is a graph showing fluorescence intensity against total lipid concentration for K-37 at three concentrations (0.4 mM, 0.65 mM and 0.9 mM) in VLDL as referred to in Example 1;

FIG. 4 is a graph showing fluorescence intensity against total lipid concentration for 0.4 mM K-37 in HDL, LDL, and VLDL as referred to in Example 1;

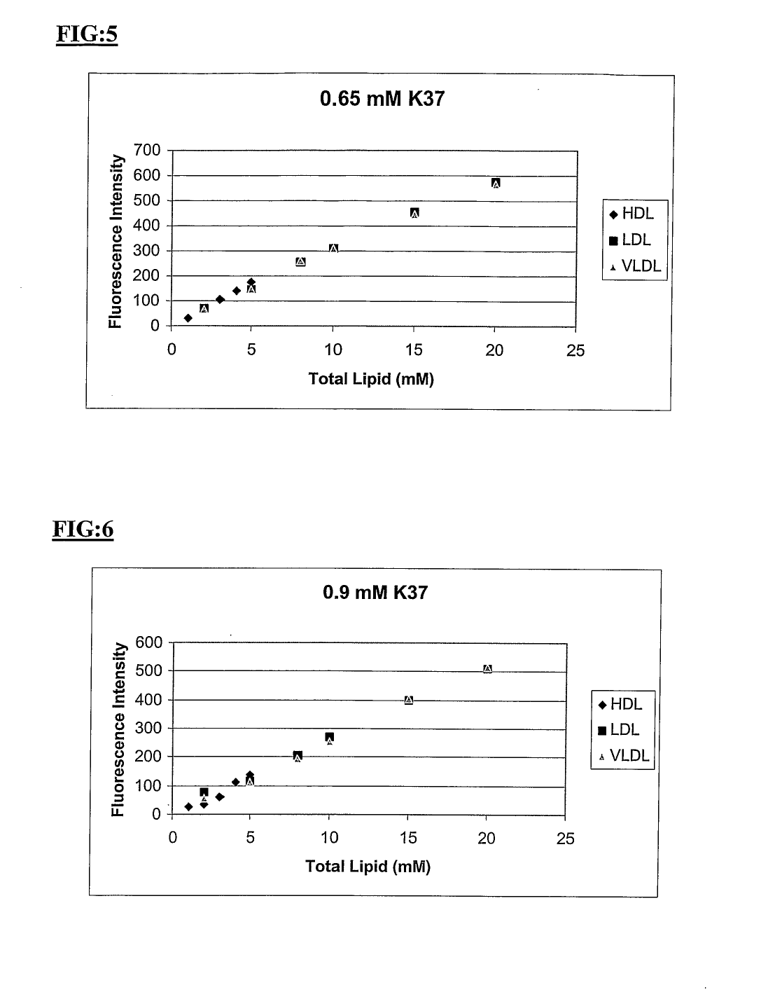

FIG. 5 is a graph showing fluorescence intensity against total lipid concentration for 0.65 mM K-37 in HDL, LDL, and VLDL as referred to in Example 1;

FIG. 6 is a graph showing fluorescence intensity against total lipid concentration for 0.9 mM K-37 in HDL, LDL, and VLDL as referred to in Example 1;

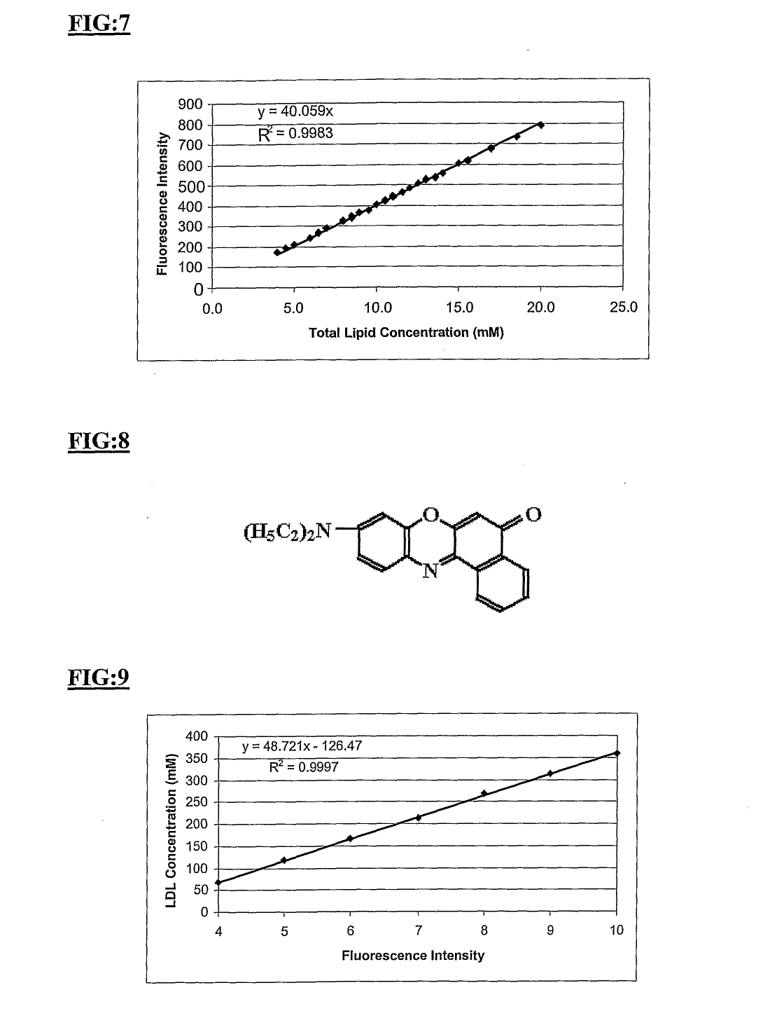

FIG. 7 is a graph showing fluorescence intensity against total lipid concentration for 0.65 mM K-37 in a series of HDL, LDL, and VLDL mixtures as referred to in Example 1;

FIG. 8 shows the structure of the dye Nile Red as referred to in description and Example 3;

FIG. 9 is a calibration curve of LDL concentration against fluorescence intensity as referred to in Example 3;

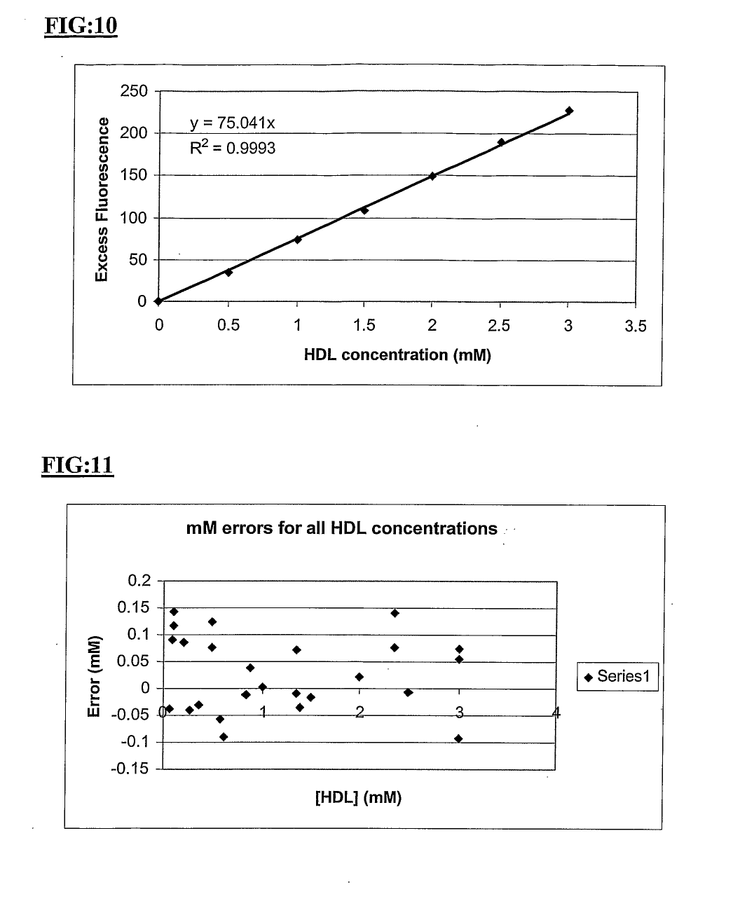

FIG. 10 is a calibration curve of excess fluorescence against HDL concentration as referred to in Example 3;

FIG. 11 is a graph showing errors against HDL concentration as referred to in Example 3;

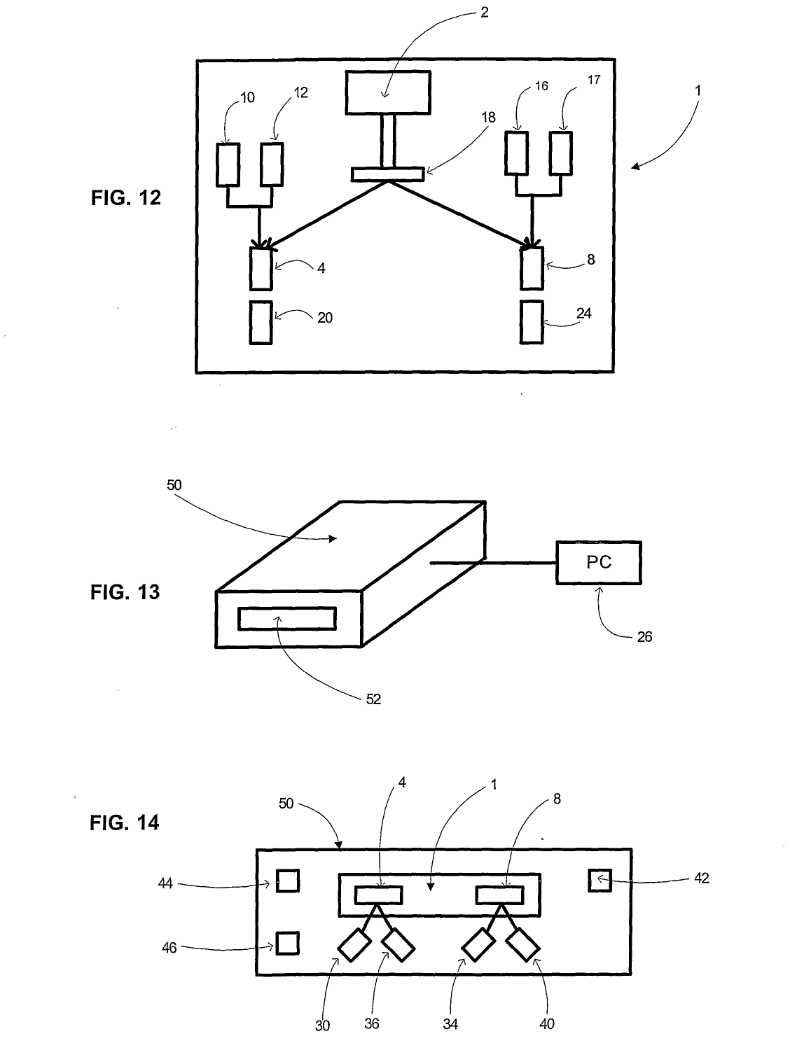

FIG. 12 shows a schematic view of an embodiment of a cartridge according to the invention as referred to in Example 5;

FIG. 13 shows a perspective view of an embodiment of a reader according to the invention as referred to in Example 5;

FIG. 14 shows a front view of the cartridge inserted in to the reader as referred to in Example 5;

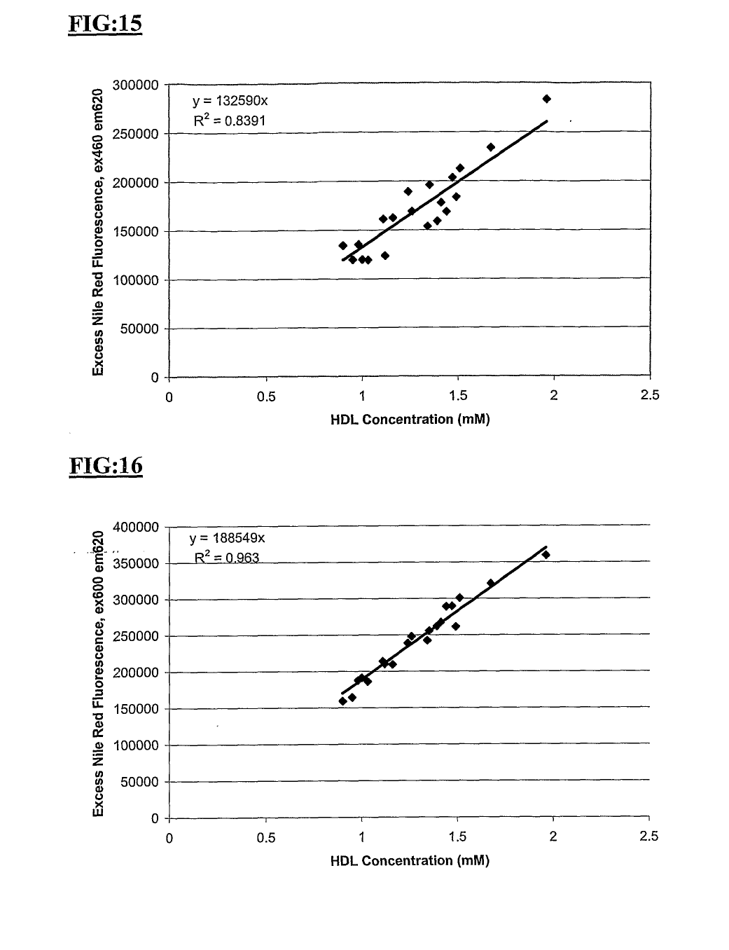

FIG. 15 is a graph illustrating Nile Red fluorescence (ex460nnm and em620nm) against HDL concentration as referred to in Example 6;

FIG. 16 is a graph illustrating Nile Red fluorescence (ex600nnm and em620nm) against HDL concentration as referred to in Example 6; and

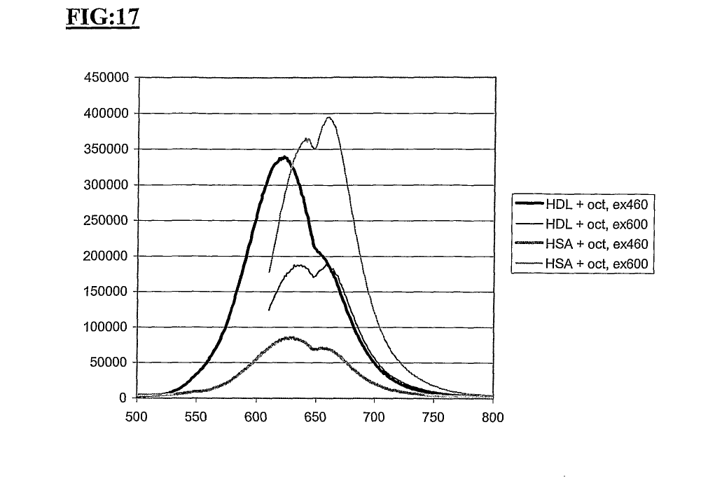

FIG. 17 is a graph illustrating a spectral analysis of Nile Red fluorescene in the presence of HDL (+octanoate) or HSA(+octanoate) at excitation wavelengths of 460 nm and 600 nm.

The inventors carried out a series of experiments in order to investigate the use of fluorescent probes to determine the concentration of total lipoproteins and also HDL in a sample, in order to generate a lipoprotein profile for a patient. Knowledge of the lipoprotein profile for a patient, is advantageous in helping a clinician decide upon a particular course of treatment. The results of these experiments, which are described in the following examples, were then used to develop the method and apparatus according to the invention.

### EXAMPLE 1

### Measurement of Total Lipoprotein Concentration

The inventors investigated the use of the fluorescent dye K-37 to detect the concentration of total lipoproteins (which equates to total triglycerides plus cholesterol plus cholesterol esters as it is assumed that all lipids are bound to lipoproteins) in a series of samples. The dye K-37 is known to the skilled technician, and is readily available. The dye is first excited at a defined wavelength, and then fluorescence is measured at another defined wavelength as described below. The intensity of the fluorescence is used to calculate the concentration of total lipoproteins in the sample (i.e. step (ii) of the method according to the invention).

### Method

The dye, K-37, which was dissolved in dimethyl formamide (DMF), was added at a range of different concentrations, to a concentration series of HDL, LDL, and VLDL dissolved in phosphate buffered saline. The objective of the experiment was to obtain a linear and equal relationship between fluorescence and lipoprotein concentration for each particle type (HDL, LDL, and VLDL), across the range of lipoprotein concentrations that would be encountered in real plasma or serum samples. Fluorescence intensity was measured in a Perkin-Elmer LS50 fluorimeter, at an excitation wavelength of 450 nm, and at an emission wavelength of 540 nm.

### Results

FIGS. 1 to 3 illustrate the fluorescence intensity versus total lipoprotein concentration for K-37 at three concentrations, i.e. 0.4 mM, 0.65 mM, and 0.9 mM, in HDL, LDL, and VLDL in phosphate buffered saline. The R² values are shown for linear fits to each series (0.4 mM at the top, 0.65 mM in the centre, and 0.9 mM below). The same data are also plotted in FIGS. 4 to 6, and are grouped by K-37 concentration.

The conclusions from the experiment are that:
1) For all three lipoprotein particle types (HDL, LDL, and VLDL), the R² shows that there is a good linear relationship between total lipoprotein concentration and fluorescence intensity at a K-37 concentration of 0.65 mM. Good linear relationships are also observed for 0.9 mM K-37 in LDL and VLDL, but the linearity at 0.9 mM K-37 in HDL is a little poorer. Linearity is poorer for all lipoproteins with 0.4 mM K-37. It is noteworthy that while it still works at concentrations where linearity is poorer, it is less accurate. However, non-linearities may be dealt with using polynomial fitting.
2) Two factors are thought to affect linearity. At a low dye concentration, there is a flattening off of the response at high total lipoprotein concentration. While the inventors do not wish to be bound by any hypothesis, they believe that this occurs because there is insufficient dye available to fully occupy the lipoprotein particles. At high dye concentrations, there is a flat response with low total lipoprotein concentration. This is caused by self-quenching of the fluorescence when the dyes are packed very closely in the particles.
3) A K-37 concentration of 0.65 mM gives linear and very similar fluorescence responses for all the lipoprotein particle types across the appropriate range when measured in phosphate buffered saline.

Accordingly, 0.65 mM K-37 was then added to a series of HDL/LDL/VLDL mixtures, and fluorescence intensity was measured as described above. The data is illustrated in FIG. 7. As can be seen, the total lipoprotein concentration is highly correlated with fluorescence intensity (R²=0.9983), confirming that this concentration of K-37 (0.65 mM) is suitable for highly accurate measurements of total lipoprotein concentration. When applying this to biological samples from patients, the inventers observed some curvature at high lipid concentration. Consequently a concentration of 0.7 mM K-37 was chosen as the optimal K-37 concentration for use in serum or plasma. Hence, this concentration was selected as the most suitable concentration for the method according to the invention.

### EXAMPLE 2

### Blocking of HSA

The inventors then conducted further investigations to optimise the method according to the invention. To this end, they realised that HSA possesses a hydrophobic binding sites in which K-37 binds and fluoresces. This additional fluorescence of K-37 when bound to HSA causes a substantial background signal, which distorts and thereby causes significant errors in the measurement of the lipoprotein molecules, i.e. HDL, LDL and VLDL. They therefore decided to block the hydrophobic binding sites in HSA with a ligand binding inhibitor, such as sodium octanoate, to see if the additional fluorescence could be minimised. It was envisaged that inhibiting the binding of K-37 with HSA in this way would improve the accuracy of the results obtained using K-37 fluorescence measurements.

### Methods

The dye K-37 was added at a concentration of 0.5 mM to LDL at a total lipid concentration of 5 mM, in the presence and absence of 50 mg/ml HSA. Measurements were made with and without the addition of 0.1 M sodium octanoate, which acted as a ligand binding inhibitor.

### Results

Fluorescence intensity was measured for all samples and is summarised in Table 1.

TABLE 1

| Sample | Fluorescence Intensity |
|---|---|
| K-37 plus 5 mM LDL | 213500 |
| K-37 plus 50 mg/ml HSA | 79300 |
| K-37 plus 5 mM LDL + octanoate | 209700 |
| K-37 plus 50 mg/ml HSA + octanoate | 3600 |

The results show that the fluorescence intensity of K-37 in LDL alone is 213500 units. The fluorescence intensity of K-37 when octanoate is added to LDL is 209700 units (i.e. about the same as without octanoate), which suggests that the presence of octanoate does not contribute to the fluorescence intensity of K-37 bound to LDL by itself. The fluorescence intensity of K-37 bound to HSA is 79300 units, whereas that of K-37 in the present of HSA and octanoate is 3600. This illustrates that HSA contributes to K-37 fluorescence and is therefore an interfering signal. The addition of octanoate significantly reduces this interference and thereby obviates the disruptive effects of HSA. The results therefore show a large suppression of fluorescence intensity for K-37 with HSA in the presence of octanoate, but little effect on K-37 fluorescence in LDL. This showed that octanoate is remarkably successful at blocking the K-37 binding site on HSA, making the K-37 fluorescence a true measure of total lipoprotein concentration.

Accordingly, the inventors believe that a ligand binding inhibitor such as octanoate, which binds the hydrophobic binding sites of HSA, can be added to the blood sample prior to measuring the fluorescence of K-37 to improve the accuracy of the total lipoprotein concentration. In addition, the inventors suggest that this technique can also be used to block the binding of other ligands to the hydrophobic binding sites of HSA, and to displace ligands that may be already bound thereto, and which have a lower affinity for HSA than the octanoate. Subsequent to this work the inventors found that 0.65 mM K-37 and 100 mM octanoate were optimal.

### EXAMPLE 3

### Measurement of HDL

The inventors then investigated whether it would be possible to distinguish between the different types of lipoprotein present in a blood sample. Hence, they tested the efficacy of using fluorescent probes, other than K-37, for example, Nile Red, to see if the lipoprotein types were distinguishable. To their surprise, they found that by using Nile Red instead of K-37, it was possible to determine the concentration of HDL in a blood sample.

### Method

The principle of the measurement is that the probe Nile Red is more fluorescent in HDL than in LDL, and VLDL, the latter having very similar but not identical fluorescence responses with concentration. The structure of Nile Red is illustrated in FIG. 8. The measurement is more complicated than the K-37 measurement for total lipoprotein, as a calculation must be made of excess fluorescence from Nile Red in HDL, and not simply total fluorescence of all lipoproteins. The procedure is as follows:

### 1) Calibration

0.5 mM Nile Red dissolved in dimethylformamide was mixed with LDL at varying total lipoprotein concentrations usually between 4 and 10 mM (typically 50 microlitres of dye are mixed with 50 microlitres of lipoprotein and 1 ml of phosphate buffered saline). Samples were put in a spectrofluorimeter and fluorescence intensity was measured (excitation wavelength 450 nm, emission wavelength 600 nM). Fluorescence intensity was plotted against LDL total lipid concentration, giving a straight calibration line with slope “X” and intercept “Y”, as shown in FIG. 9.

The procedure was then repeated for mixtures of LDL and HDL. HDL was added at concentrations of between 0 and 3.0 mM, with LDL added to keep the total lipoprotein concentration at 6 mM for all samples (but 3-12 mM would be the limits). Fluorescence intensities for these samples were then measured. A plot was then made of excess fluorescence due to the presence of HDL, giving a straight calibration line having slope “Z”, as illustrated in FIG. 10.

### 2) Measurements of Unknowns

0.5 mM Nile Red dissolved in dimethylformamide was mixed with the sample under investigation. The sample was put into a fluorimeter and fluorescence intensity was measured under the same conditions as for the calibration described above.

### 3) Calculation of HDL Concentration

Calculation of HDL requires knowledge of the total lipoprotein concentration “A”, which can for example but not exclusively be measured from the fluorescence intensity of K-37. For a particular sample, the fluorescence intensity that would be expected if the sample contained no HDL is obtained from the calibration line shown in FIG. 10. The measured fluorescence intensity minus this calculated fluorescence intensity is the excess fluorescence due to HDL present in the sample.

The HDL concentration “C” in the unknown sample can then be obtained using the calibration line shown in FIG. 10 and the following equation:

C=(B−(AX−Y))/Z

A range of concentrations of HDL/LDL/VLDL mixtures were prepared intended to cover the range of concentrations that would be expected in real clinical samples. The calibration data discussed above were used to calculate HDL concentrations from the mixtures. FIG. 11 illustrates errors between actual HDL concentration and HDL concentration determined from Nile Red fluorescence, showing a maximum error of only approximately 0.15 mM. The inventors further refined the concentration of Nile Red for use in samples of serum to be 0.4 mM.

As a result of these data, the inventors have shown that it is possible to distinguish between the types of lipoprotein present in a sample, and to determine the concentration of HDL using the dye Nile Red.

Following the findings described in Example 2, concerning the addition of octanoate to block the hydrophobic binding sites of HSA, the inventors then observed that Nile Red also binds to HSA and fluoresces. This additional fluorescence of Nile Red when bound to HSA also causes a substantial background signal, which distorts and thereby causes significant errors in the measurement of HDL. They therefore decided to block the hydrophobic binding sites in HSA with the same ligand binding inhibitor as for K-37 blocking, i.e. sodium octanoate. The experiments conducted with Nile Red and HSA, were based on those discussed in Example 2, and all using 0.5 mM Nile Red.

TABLE 2

| Sample | Fluorescence Intensity |
|---|---|
| Nile Red plus 5 mM LDL | 187.532 |
| Nile Red plus 50 mg/ml HSA | 58.905 |
| Nile Red plus 5 mM LDL + 50 mM | 183.786 |
| octanoate | |
| Nile Red plus 50 mg/ml HSA + 50 mM | 9.118 |
| octanoate | |
| PBS + 50 mM Octanoate | 7.382 |

The results presented in Table 2 show that the fluorescence intensity of Nile Red in LDL alone is 187.532 units. The fluorescence intensity of Nile Red when octanoate is added to LDL is 183.786 units (i.e. about the same as without octanoate), which suggests that the presence of octanoate does not contribute to the fluorescence intensity of K-37 bound to LDL by itself. The fluorescence intensity of Nile Red bound to HSA is 58.905 units, whereas that of Nile Red in the present of HSA and octanoate is 9.118. This illustrates that HSA contributes to Nile Red fluorescence and is therefore an interfering signal. The addition of octanoate significantly reduces this interference and thereby obviates the disruptive effects of HSA. The results therefore show a large suppression of fluorescence intensity for Nile Red with HSA in the presence of octanoate, but little effect on Nile Red fluorescence in LDL.

This showed that octanoate is remarkably successful at blocking the Nile Red binding site on HSA, making the Nile Red fluorescence a true measure of lipoprotein concentration. Accordingly, the inventors believe that a ligand binding inhibitor such as octanoate, which can fit in the hydrophobic binding sites of HSA, can be added to the blood sample prior to measuring the fluorescence of Nile Red to improve the accuracy of the lipoprotein (HDL) concentration. Subsequent to this work the inventors found that 0.4 mM Nile Red and 50 mM, or more preferably about 10 mM, octanoate were optimal for the analysis of serum samples.

Example 6 describes further development work whereby fluorescent measurements with Nile Red may be further improved by addition of an agent that binds to the drug binding domain of HSA (e.g. benzoic acid).

### EXAMPLE 4

### Simultaneous Assay to Generate Lipoprotein Profile

Examples 1 and 3 illustrate how fluorescence measurements of the dye K37 may be used to determine the concentration of total lipoproteins in a sample, and how the fluorescence measurements of Nile Red may be used to determine the concentration of HDL in a sample. Example 2 describes blocking HSA to improve the accuracy of the results produced by K-37 fluorescence.

Therefore, the inventors realised that it is possible to create a single parallel method for analysing the lipid composition of a patient's blood sample in order to create a lipoprotein profile for that patient. The preferred method consists of two assays, both of which can be carried out under very similar conditions, and hence, can produce results very quickly. The clinician may use this information to decide upon a course of treatment.

### Method

A blood sample is initially taken from a patient, and then centrifuged using well-established conventional techniques, in order to separate the serum. The serum is then separated in to two 1 ml aliquots (a, & b), each of which is subjected to biochemical analysis to determine the concentration of a lipid component. Aliquot (a) is used to determine the concentration of total lipoprotein; and aliquot (b) is used to determine the concentration of HDL, as described below.

Aliquot (a)—The HSA ligand binding inhibitor, sodium octanoate, is added to the 1 ml of serum to a concentration of 100 mM as described in Example 2 above. The probe K-37, which was dissolved in dimethyl formamide (DMF), was then slowly added under stirring to the sample to a final concentration of 0.7 mM. The sample was then excited at about 450 nm in order to cause the probe to fluoresce. The fluorescence was measured at an emission wavelength of above 520 nm or 540 nm, and from this value it was then possible to determine the concentration of total lipoprotein (HDL, LDL, and VLDL) in the sample, as described in Example 1 above.

Aliquot (b)—The HSA ligand binding inhibitor, sodium octanoate, is added to the 1 ml of serum to a concentration of 50 mM or 100 mM as described in Example 3 above. Furthermore benzoic acid may be added to the serum to a concentration of 5 mM. The probe Nile Red was then slowly added under stirring to the sample to a final concentration of 0.4 mM. The sample was then excited at 600 nm in order to cause the probe to fluoresce. The fluorescence was measured at an emission wavelength of 620 nm, and from this value it was then possible to determine the concentration of HDL in the sample as described in Example 3 above.

### EXAMPLE 5

A device for generating lipoprotein profile Referring to FIGS. 12-14, there is shown a portable device developed by the inventors, which can be used to generate a patient's lipoprotein profile. The device consists of a cartridge 1, which is shown in detail in FIG. 12, and a reader 50, which is shown in detail in FIGS. 13 and 14.

The cartridge 1 has a series of interconnected reservoirs, along which fluids may flow in order to carry out the assays according to the invention. The cartridge 1 plugs into the reader 50 via slot 52 for detecting and measuring fluorescence intensity for each of the assays carried out in the cartridge 1.

Referring to FIG. 12, the cartridge 1 has a sample reservoir 2 in which a biological fluid taken from a patient, such as blood, is contained. A filter 18 is provided for removing blood cells from the blood, leaving lymph or serum or other body fluid, with which the assays are carried out. The fluid is divided in to two aliquots (a and b, as described in Example 4), and urged along channels in to reaction reservoirs 4, 8, respectively.

Two reagent reservoirs 10, 12 containing K37 and sodium octanoate, respectively, are connected to reservoir 4 (aliquot (a)). The dye and octanoate are urged in to reaction reservoir 4 and the total lipoprotein assay is initiated.

Two reagent reservoirs 16, 17 containing Nile Red; and sodium octanoate+optionally benzoic acid, respectively, are connected to reaction reservoir 8 (aliquot (b)). The Nile Red and octanoate are urged in to reservoir 8, mixed with the fluid, and the HDL assay is initiated. It will be appreciated that it may be possible to have just one reagent reservoir for containing sodium octanoate instead of two reservoirs, 12, 17, and that the octanoate could be fed from this one reservoir into reaction reservoirs 4, 8 as required. The cartridge may also include fluorescence standards 20, 24, which may be used for calibrating the reader 50.

In one embodiment of the apparatus (i.e. the cartridge 1 and the reader 50), the two assays are carried out in the reaction reservoirs 4, 8. The cartridge 1 plugs into the slot 52 in the front of the reader 50, as shown in FIG. 13. Slotting the cartridge 1 in to the reader 50 causes the two reaction reservoirs 4, 8 to each align with a corresponding excitation means (light sources 30, 34) and a corresponding detection means (photodiodes 36, 40), which are present in the reader 50. In another embodiment, the reader 50 has only one light source or LED instead of separate LEDs for each reservoir 4, 8.

The LEDs (or guides from an LED) 30, 34 each provide the corresponding assay with the required fluorescence excitation illumination for each assay to fluoresce. The wavelength of the light from each of the LEDs 30, 34 is at about 450-470 nm and also for some embodiments of the assay may be about 600 nm. The light may pass through a 450 nm or 600 nm interference filter (not shown) before it is directed to the reaction reservoirs 4, 8. The reader 50 may have an excitation correction system 46. Hence, fluorescence of the two assays (a, and b) is collected with lenses or similar collection optics, and may pass through a polariser at a wavelength of 540 nm for assay a (total lipoprotein) and at a wavelength of about 600 nm or 620 nm for assay b (HDL). Alternatively a common polariser may be used for both assays. For measurement of the fluorescence intensity, the current output of the photodiodes 36, 40 is amplified and read as a current or a voltage.

Accordingly, the reader 50 is configured to hold the cartridge 1 to detect and measure the fluorescence intensities of each of the two assays, to thereby determine the total lipoprotein and HDL concentration. In one embodiment, the apparatus has an LCD readout display 42 on which the concentrations of each of the blood components are shown. In another embodiment the reader 50 may be powered by, and have its readout fed through, a USB port of a PC, laptop, or PDA 26 enabling the clinician to readout information on the concentrations of lipoproteins. Alternatively, the apparatus may embody both aspects of the cartridge and measurement instrument and comprise a microprocessor 44 which can calculate the concentrations of each of the lipid components itself.

Advantages of the cartridge 1 and reader device 50 reside in the quick and easy assay systems, which can be carried out simultaneously to generate the lipoprotein profile from the biological fluid. A clinician can then decide on an effective course of treatment. The cartridge 1 is disposable and may be cheaply made being prepared with the reagents for both assays.

### EXAMPLE 6

Further optimisation of the assay according to the invention Further tests were performed on human serum samples to investigate optimum excitation wavelengths for inducing fluorescence indicative of HDL levels according to the method of the invention.

The inventors tested a number of wavelengths and have established that, when using Nile Red, that an excitation wavelength of 600 nm and an emission wavelength of 620 nm gives optimal results (see FIG. 15). The inventors were surprised that this excitation wavelength was optimal because it is to the very long wavelength edge of the spectrum.

For certain samples the inventors observed a noisier plot with an excitation wavelength of 460 nm and an emission wavelength of 620 nm (see FIG. 16).

The inventors believe that Nile Red is about 5 times more fluorescent in HDL than VLDL and LDL when excited at 600 nm as opposed to excitation at 460 nm where it is only about 2 times more fluorescent. This gives a better signal to noise when subtracting from the standard curve of LDL plus VLDL.

Although the inventors do not wish to be bound by any hypothesis, they believe the “noise” observed in serum samples, excited at 460 nm, is an effect of signal-to-noise. The inventors have noted that Nile Red binds to HSA and particularly at low lipid concentrations. They therefore performed a spectral analysis of Nile Red fluorescene in the presence of HDL (+octanoate) or HSA(+octanoate) both at an excitation wavelength of 460 nm and 600 nm (see FIG. 17). These experiments resulted in unexpected spectral behaviour which the inventors believe may be explained by the fact that Nile red is in a rigid but polar environment (binding site on HSA) and the Nile red exhibits twisted intramolecular charge transfer (TICT) (Journal of Photochem and Photobiol A: Chemistry 93 (1996) 57-64) that shifts the excitation and emission to longer wavelengths. The molecule in this excited state has a different dipole moment and so behaves like a different species. In exciting at 600 nm the better signal-to-noise due to the larger difference in signal between Nile Red in HDL compared with other lipoproteins more than compensates for the excitation of the TICT state because TICT fluorescence is excluded by the 620 nm emission wavelength setting. In other words, while we excite the HSA/NileRed more optimally at 600 nm its fluorescence is rejected by the instrument.

This lead the inventors to realise that the HDL/Nile red assay may be improved further by using an additional blocker. They tried agents that block the drug binding domain of HSA. To their surprise they found that agents such as benzoic acid, and its trichoro and triiodo derivatives, all worked to displace the Nile Red from HSA without affecting the lipoprotein fluorescence at about 5 mM. The benzoic acid has the added bonus of quenching the Nile Red residual fluorescence in solution by about 20%.

1. A method of determining the concentration of total lipoprotein in sample solution, the method comprising the steps of:
(i) adding to a first aliquot of the sample between 0.2 mM-1.0 mM of a probe substance, K-37 (defined herein), which probe substance binds to lipoproteins in the sample, and which when so bound fluoresces under appropriate excitation; and
(ii) determining the total lipoprotein concentration in the sample using fluorescence analysis.

2. The method according to claim 1, wherein the concentration of K-37 added to the first aliquot is between approximately 0.5-0.85 mM.

3. The method according to claim 1 or 2, wherein the method comprises adding to the first aliquot a ligand binding inhibitor that is adapted to substantially inhibit the binding of the probe substance to a hydrophobic binding domain on Human Serum Albumin before lipoprotein or HDL concentrations are determined.

4. The method according to claim 3, wherein the ligand binding inhibitor comprises a fatty acid or a functional derivative thereof.

5. The method according to either claim 3 or claim 4, wherein the ligand binding inhibitor comprises octanoic acid (C₈) or a derivative thereof, for example, octanoate.

6. The method according to any preceding claim, wherein, for each step, fluorescence is induced by exciting the first aliquot at an excitation wavelength of between 400 nm-650 nm, and the resultant fluorescence is measured at an emission wavelength of between about 540 nm-700 nm.

7. The method according to any preceding claim, wherein the method further comprises the step of determining the concentration of a particular class or subclass of lipoprotein.

8. The method according to claim 7 wherein a second probe substance is added to a second aliquot of the sample; wherein the second probe binds to a specific class or subclass of lipoproteins such that when it is bound thereto it modifies the fluorescence yield under appropriate excitation; and this fluorescence is indicative of the concentration of the specific class or sub-class of lipoproteins.

9. The method according to claim 8, wherein the class or subclass of lipoprotein is HDL.

10. The method according to claim 9, wherein the second probe substance is Nile Red.

11. The method according to claim 10, wherein the concentration of the probe substance Nile Red added to the sample is between approximately 0.1-0.9 mM.

12. The method according to any one of claims 8-11 wherein a ligand binding inhibitor according to any one of claims 5-7 is added to the second aliquot.

13. The method according to any one of claims 8-12 wherein an agent that blocks the drug binding domain of HSA is also added to the second aliquot before HDL concentrations are determined.

14. The method according to claim 13 wherein the agent is benzoic acid or a salt or derivative thereof.

15. The method according to any one of claims 10-14 wherein fluorescence is induced by: exciting the second aliquot at an excitation wavelength of between 400 nm-650 nm and measuring the resultant fluorescence at an emission wavelength of between about 540 nm-700 nm.

16. The method according to any one of claims 10 -15 wherein fluorescence is induced by: exciting the first aliquot at an excitation wavelength of between 400 nm-500 nm; exciting the second aliquot at an excitation wavelength of about 600 nm; and measuring the resultant fluorescence from each aliquot at an emission wavelength of about 620 nm.

17. The method according to any preceding claim, wherein the sample is a biological fluid.

18. The method according to any preceding claim, wherein the sample comprises blood plasma or serum, or lymph.

19. An apparatus, for conducting a lipoprotein assay according to any one of claims 1-18, comprising: a reaction reservoir; containment means adapted to contain reagents required for the method according to any one of claims 1-18; excitation means operable to excite the sample so that it fluoresces, and detection means operable to detect the fluorescence emitted by the sample.

20. The apparatus according to either claim 19, wherein the apparatus comprises a reader and a cartridge, which cartridge comprises the reaction reservoir, and the containment means.

21. The apparatus according to claims 19 or 20, wherein the excitation means comprises an illumination source operable to illuminate the sample at about 460 nm and also optionally at 600 nm.

22. The apparatus according to any one of claims 19-21, wherein the detection means detects fluorescence emitted by the sample at about 500 nm-700 nm.
